(19) United States
(12) Patent Application Publication
(10) Pub. No.: US 2023/0011154 A1
(43) Pub. Date: Jan. 12, 2023

(54) PORTABLE ELECTROCARDIOGRAPHIC DEVICE, ELECTROCARDIOGRAM MEASUREMENT SYSTEM, AND NON-TRANSITORY RECORDING MEDIUM HAVING PROGRAM RECORDED THEREIN

(71) Applicant: OMRON HEALTHCARE Co., Ltd., Kyoto (JP)

(72) Inventor: Mitsuru SAMEJIMA, Kyoto (JP)

(21) Appl. No.: 17/932,159

(22) Filed: Sep. 14, 2022

Related U.S. Application Data
(63) Continuation of application No. PCT/JP2021/009346, filed on Mar. 9, 2021.

(30) Foreign Application Priority Data
Mar. 19, 2020 (JP) 2020-048879

(51) Int. Cl. A61B 5/024; A61B 5/00

(52) U.S. Cl. CPC A61B 5/02438; A61B 5/746; A61B 5/742; A61B 5/7282

(57) ABSTRACT

A portable electrocardiographic device configured to measure an electrocardiographic waveform using a plurality of types of lead systems includes an electrode unit configured to be brought into contact with a subject's body and measure an electrocardiographic waveform, an analysis unit configured to analyze the electrocardiographic waveform measured by the electrode unit in accordance with a lead system at a time of measurement of the electrocardiographic waveform, a storage unit configured to store the electrocardiographic waveform measured at the electrode unit, the lead system, and an analysis result of the electrocardiographic waveform analyzed by the analysis unit in association with one another, and a remeasurement facilitating unit configured to prompt a user, when the analysis result or a state of the measured electrocardiographic waveform satisfies a predetermined condition, for remeasurement in a predetermined lead system different from the lead system at the time of the measurement of the electrocardiographic waveform.

### CROSS-REFERENCE TO RELATED APPLICATIONS

This application is the U.S. national stage application filed pursuant to 35 U.S.C. 365(c) and 120 as a continuation of International Patent Application No. PCT/JP2021/009346, filed Mar. 9, 2021, which application claims priority to Japanese Patent Application No. 2020-048879, filed Mar. 19, 2020, which applications are incorporated herein by reference in their entireties.

### TECHNICAL FIELD

The present invention relates to a portable electrocardiographic device and an electrocardiographic measurement system including the portable electrocardiographic device that allow electrocardiographic waveform measurement in daily life.

### BACKGROUND ART

Portable electrocardiographic measurement devices (hereinafter also referred to as “portable electrocardiographic devices”) have been proposed that allow immediate electrocardiographic waveform measurement upon occurrence of abnormalities such as chest pain and palpitation in daily life. When symptoms such as palpitation occur at home, outside the home, or the like, a physician or the like can detect a heart disease at an early timing and perform appropriate treatment based on data on the electrocardiographic waveform measured by the electrocardiographic device.

Conventionally, for such portable electrocardiographic devices, there is an international agreement on methods of recording electrocardiographic waveforms (lead systems and lead types), and a lead system that uses a plurality of types of lead systems is widely used. This lead system based on the international agreement includes six types of limb leads and six types of chest leads, and electrocardiographic waveforms are detected and recorded using appropriate lead systems.

Among these, for a lead system known as lead I in which a contact unit including a positive electrode is pressed against the left hand of the subject and a lead system known as lead V4 in which the contact unit including the positive electrode is pressed against the left chest of the subject, a technology of displaying a measurement result such as an electrocardiographic waveform on the display unit in an easy-to-see manner is known (e.g., See Patent Document 1; Citation, JP 2005-000468 A). More specifically, this technology displays the measurement result in the lateral direction on the display unit when measurement is performed with lead I, and displays the measurement result in the vertical direction on the display unit when measurement is performed in lead V4.

### SUMMARY OF INVENTION

### Technical Problem

However, in the conventional technology described above, during electrocardiographic waveform measurement, the optimum lead system corresponding to the state of the electrocardiographic waveform is not necessarily used, and thus the quality of the electrocardiographic waveform and the accuracy of the analysis result is deteriorated in some cases.

In view of the problems described above, an object of the present invention is to provide a technology that, when measuring an electrocardiographic waveform, allows measurement to be made using the optimal lead system corresponding to the state of the electrocardiographic waveform, and that improves the accuracy of electrocardiographic measurement.

### Solution to Problem

To solve the problems described above, an aspect of the present invention is a portable electrocardiographic device configured to measure an electrocardiographic waveform using a plurality of types of lead systems, the portable electrocardiographic device including an electrode unit configured to be brought into contact with a predetermined location of a subject's body and measure an electrocardiographic waveform, an analysis unit configured to analyze the electrocardiographic waveform measured by the electrode unit in accordance with a lead system at a time of measurement of the electrocardiographic waveform, a storage unit configured to store the electrocardiographic waveform measured at the electrode unit, the lead system, and an analysis result of the electrocardiographic waveform analyzed by the analysis unit in association with one another, and a remeasurement facilitating unit configured to prompt a user, when the analysis result or a state of the measured electrocardiographic waveform satisfies a predetermined condition, for remeasurement in a predetermined lead system different from the lead system at the time of the measurement of the electrocardiographic waveform.

Here, when an electrocardiographic waveform is measured in a specific lead system, the measurement is not performed in the lead system optimal for the analysis result and the state of the electrocardiographic waveform in some cases. As described above, when the measurement is not performed in the optimal lead system, the accuracy of the analysis result is also deteriorated. In contrast, in the present invention, when the analysis result or the state of the electrocardiographic waveform satisfies a predetermined condition, the remeasurement facilitating unit prompts for remeasurement with the lead system changed to the optimal one. Even when the lead system used at the time of initial measurement was not optimal, this allows remeasurement to be performed with the lead system optimized. As a result, the accuracy of analysis result can be improved. Here, the user refers to a person who operates the portable electrocardiographic device.

Furthermore, in an aspect of the present invention, the remeasurement facilitating unit may include a display unit that displays the lead system to be set at the time of the remeasurement. In this case, for example, light-emitting units each associated with a corresponding lead system of the plurality of types of lead systems may be provided at the device main body, and the light-emitting unit associated with the lead system to be set at the time of remeasurement may be caused to emit light. Alternatively, a display unit capable of displaying characters may directly display the lead system to be set at the time of remeasurement. This allows the user to recognize the lead system to be set at the time of remeasurement more easily.

Furthermore, in an aspect of the present invention, the display unit may further display indicating that the predetermined condition is satisfied. This allows the user to more easily recognize the reason why the lead system to be set at the time of remeasurement is selected.

Furthermore, in an aspect of the present invention, a setting unit configured to set lead system used in measurement of the electrocardiographic waveform, among the plurality of types of lead systems, may be further included, and at the time of the measurement and at the time of the remeasurement, the user may set the lead system using the setting unit. This allows the user to select the lead system to be used at the time of measurement or at the time of remeasurement of his or her own volition.

Furthermore, in an aspect of the present invention, the lead system at the time of the measurement may be lead I in the 12-lead system, the predetermined condition may be that arrhythmia is observed in the analysis result, and the predetermined lead system may be lead V4 in the 12-lead system.

Here, it has been found that the waveform that characterizes arrhythmia is difficult to detect with lead I in the 12-lead system, and that arrhythmia can be more accurately analyzed by measuring with lead V4. In other words, in electrocardiographic waveforms with lead I, it is relatively easy to detect arrhythmia based on the interval between R waves, but it is difficult to detect arrhythmia based on waveform shapes other than that of R waves. On the other hand, when an electrocardiographic waveform with a lead system other than lead I (e.g., lead V4) is used, arrhythmia other than that in the waveform rhythm can be more accurately detected. Thus, in the present invention, an electrocardiographic waveform is measured using lead I at the time of measurement, and when arrhythmia is observed in the analysis result, the remeasurement facilitating unit facilitates remeasurement using lead V4. This allows arrhythmia to be more accurately diagnosed.

Furthermore, in an aspect of the present invention, the lead system at the time of the measurement may be lead I in the 12-lead system, the predetermined condition may be that atrial fibrillation is observed in the analysis result, and the predetermined lead system may be lead V1 in the 12-lead system.

Here, it has been found that the waveform that characterizes atrial fibrillation is difficult to detect with lead I in the 12-lead system, and that atrial fibrillation can be more accurately analyzed by measuring with lead V1. In other words, while the characteristics of atrial fibrillation include RR interval irregularity, loss of P waves, and appearance of F waves, the characteristics other than RR interval irregularity are difficult to capture in the electrocardiographic waveform with lead I, but are easy to capture in the electrocardiographic waveform with lead V1. Thus, in the present invention, an electrocardiographic waveform is measured using lead I at the time of measurement, and when atrial fibrillation is observed in the analysis result, the remeasurement facilitating unit facilitates remeasurement using lead V1. This allows atrial fibrillation to be more accurately diagnosed.

Furthermore, in an aspect of the present invention, the lead system at the time of the measurement may be lead I in the 12-lead system, the predetermined condition may be that a defect in waveform quality is observed in the analysis result, and the predetermined lead system may be lead V1 or lead V4 in the 12-lead system.

Here, it has been found that defects in waveform quality of the electrocardiographic waveform are difficult to detect with lead I in the 12-lead system, and that defects in waveform quality of the electrocardiographic waveform can be more accurately analyzed by measuring with lead V1 or lead V4. Thus, in the present invention, an electrocardiographic waveform is measured using lead I at the time of measurement, and when a defect in waveform quality is observed in the analysis result, the remeasurement facilitating unit facilitates remeasurement using lead V1 or lead V4. This allows defects in waveform quality of the electrocardiographic waveform to be more accurately detected.

Furthermore, an aspect of the present invention may be an electrocardiographic measurement system configured to measure an electrocardiographic waveform using a plurality of types of lead systems, the electrocardiographic measurement system including a portable electrocardiographic device provided with an electrode unit configured to be brought into contact with a predetermined location of a subject's body and detect an electrocardiographic waveform, and a portable communication terminal provided communicably with the portable electrocardiographic device, wherein the electrocardiographic measurement system further includes an analysis unit configured to analyze the electrocardiographic waveform measured by the electrode unit in accordance with a lead system at a time of measurement of the electrocardiographic waveform, a storage unit configured to store the electrocardiographic waveform measured at the electrode unit, the lead system, and an analysis result of the electrocardiographic waveform analyzed by the analysis unit in association with one another, and a remeasurement facilitating unit configured to prompt a user, when the analysis result or a state of the measured electrocardiographic waveform satisfies a predetermined condition, for remeasurement in a predetermined lead system different from the lead system at the time of the measurement of the electrocardiographic waveform. Here, the user refers to a person who operates the electrocardiographic measurement system.

Furthermore, an aspect of the present invention may be the electrocardiographic measurement system described above, wherein the remeasurement facilitating unit includes a display unit provided at either the portable electrocardiographic device or the portable communication terminal, the display unit is configured to display the lead system to be set at a time of the remeasurement.

This allows the user to recognize the lead system to be used at the time of remeasurement using the display unit. Furthermore, when the display unit is provided at the portable communication terminal, the lead system to be used at the time of remeasurement can be displayed using the high-performance display of the portable communication terminal.

Furthermore, an aspect of the present invention may be the electrocardiographic measurement system described above, wherein the display unit further displays indicating that the predetermined condition is satisfied.

This allows the user to recognize about the predetermined condition using the display unit. Furthermore, when the display unit is provided at the portable communication terminal, the predetermined condition can be displayed using the high-performance display of the portable communication terminal.

Furthermore, an aspect of the present invention may be the electrocardiographic measurement system described above, the electrocardiographic measurement system further including a setting unit configured to set lead system used in measurement of the electrocardiographic waveform, among the plurality of types of lead systems, wherein at the time of the measurement and at a time of the remeasurement, the user sets the lead system using the setting unit.

This allows the lead system used at the time of measurement and at the time of remeasurement to be set. Furthermore, when the setting unit is provided at the portable communication terminal, the lead system used at the time of measurement and at the time of remeasurement can be remotely set using the portable communication terminal.

Furthermore, an aspect of the present invention may be the electrocardiographic measurement system described above, wherein the lead system at the time of the measurement is lead I in a 12-lead system, the predetermined condition is that arrhythmia is observed in the analysis result, and the predetermined lead system is lead V4 in the 12-lead system.

Furthermore, an aspect of the present invention may be the electrocardiographic measurement system described above, wherein the lead system at the time of the measurement is lead I in a 12-lead system, the predetermined condition is that atrial fibrillation is observed in the analysis result, and the predetermined lead system is lead V1 in the 12-lead system.

Furthermore, an aspect of the present invention may be the electrocardiographic measurement system described above, wherein the lead system at the time of the measurement is lead I in a 12-lead system, the predetermined condition is that a defect in waveform quality is observed in the analysis result, and the predetermined lead system is lead V1 or lead V4 in the 12-lead system.

Furthermore, when the display unit in the electrocardiographic measurement system described above is provided at the portable communication terminal, an aspect of the present invention may be a non-transitory recording medium having recorded therein a program for controlling the portable communication terminal to cause the display unit to display the lead system to be set at the time of the remeasurement.

Furthermore, when the display unit in the electrocardiographic measurement system described above is provided at the portable communication terminal, an aspect of the present invention may be a non-transitory recording medium having recorded therein a program for controlling the portable communication terminal to cause the display unit to display indicating that the predetermined condition is satisfied.

Furthermore, when the setting unit in the electrocardiographic measurement system described above is provided at the portable communication terminal, an aspect of the present invention may be a non-transitory recording medium having recorded therein a program for controlling the portable communication terminal to allow a user to set the lead system using the setting unit at the time of the measurement and at the time of the remeasurement.

Note that in the present invention, the unit to solve the problems described above can be used in combination as long as such combination is practicable.

### Advantageous Effects of Invention

According to the present invention, when measuring an electrocardiographic waveform, measurement can be made using the optimal lead system corresponding to the state of the electrocardiographic waveform, and the accuracy of electrocardiographic measurement can be improved.

### BRIEF DESCRIPTION OF DRAWINGS

FIG. 1(A) to FIG. 1(F) are each a view illustrating an external appearance of a portable electrocardiographic device according to the present embodiment.

FIG. 2 is a functional block diagram of the portable electrocardiographic device according to the present embodiment.

FIG. 3 is a functional block diagram of a smartphone according to the present embodiment.

FIG. 4 is a flowchart illustrating procedures for basic electrocardiographic measurement processing of the portable electrocardiographic device according to the present embodiment.

FIG. 5 is a diagram illustrating an electrocardiographic waveform and identifying parameters.

FIG. 6(A) to FIG. 6(L) are each a diagram illustrating an example of an electrocardiographic waveform for each of the lead types.

FIG. 7 is a flowchart illustrating procedures for electrocardiographic measurement processing in which a different lead system is added in the portable electrocardiographic device according to the present embodiment.

FIG. 8 is a portion of a flowchart illustrating procedures for basic electrocardiographic measurement processing in which the portable electrocardiographic device and the smartphone according to the present embodiment cooperate with each other.

FIG. 9 is a portion of a flowchart illustrating procedures for basic electrocardiographic measurement processing in which the portable electrocardiographic device and the smartphone according to the present embodiment cooperate with each other.

FIG. 10(A) and FIG. 10(B) are each a view illustrating a display example of the smartphone according to the present embodiment.

FIG. 11 is a portion of a flowchart illustrating procedures for electrocardiographic measurement processing in which the portable electrocardiographic device and the smartphone according to the present embodiment cooperate with each other to add a different lead system.

FIG. 12 is a portion of a flowchart illustrating procedures for electrocardiographic measurement processing in which the portable electrocardiographic device and the smartphone according to the present embodiment cooperate with each other to add a different lead system.

FIG. 13(A) and FIG. 13(B) are each a view illustrating another display example of the smartphone according to the present embodiment.

### DESCRIPTION OF EMBODIMENTS

Embodiments of the present invention will be specifically described below with reference to the drawings. Hereinafter, an example of the embodiments of the present invention will be described. It should be noted, however, that the dimension, material, shape, relative arrangement, and the like of the components described in the present embodiment are not intended to limit the scope of this invention to them alone unless otherwise stated.

### First Embodiment

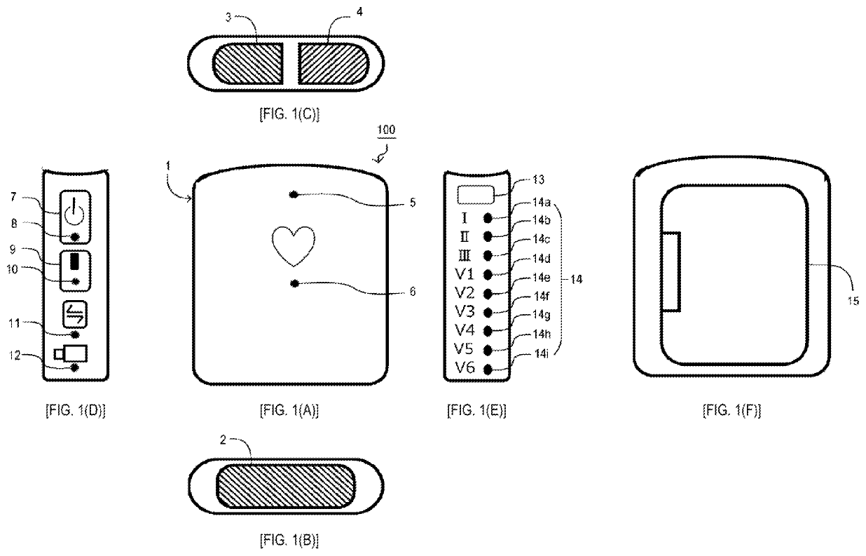

Configuration of Portable Electrocardiographic Device. FIG. 1(A) to FIG. 1(F) are each a view illustrating an example of a configuration of a portable electrocardiographic device 100 according to the present embodiment. FIG. 1(A) is a view of the portable electrocardiographic device 100 as viewed from the front surface. FIG. 1(B) is a view of the portable electrocardiographic device 100 as viewed from below. FIG. 1(C) is a view of the portable electrocardiographic device 100 as viewed from above. FIG. 1(D) is a view illustrating the left side surface as viewed from the front surface of the portable electrocardiographic device 100. FIG. 1(E) is a view illustrating the right side surface as viewed from the front surface of the portable electrocardiographic device 100. FIG. 1(F) is a view of the portable electrocardiographic device 100 as viewed from the rear surface. The vertical direction refers to the vertical direction on the paper surface relative to the portable electrocardiographic device 100 having the posture illustrated in FIG. 1(A).

As illustrated in FIG. 1(A) to FIG. 1(F), the main body 1 of the portable electrocardiographic device 100 has the shape of a substantially quadrangular prism with rounded corners, and is formed to be flat between the front surface and the rear surface. A first electrode 2 is provided at the bottom of the portable electrocardiographic device 100. At the top of the portable electrocardiographic device 100, a second electrode 3 is provided on the left side and a third electrode 4 is provided on the right side as viewed from the front surface. The top of the portable electrocardiographic device 100 has a smoothly curved shape so that the right hand index finger of the subject is easily brought into contact therewith.

At the front surface of the main body 1 of the portable electrocardiographic device 100, a measurement notification LED 5 and an abnormal waveform detection LED 6 are disposed vertically side by side. The measurement notification LED 5 is a light-emitting element that is turned on or blinks during electrocardiographic waveform measurement. The abnormal waveform detection LED 6 is a light-emitting element that is turned on when an abnormal waveform is detected for the measured electrocardiographic waveform. Through the turning on of the abnormal waveform detection LED 6, the subject is notified of the presence or absence of an abnormal waveform detected from electrocardiographic waveform measurement data.

On the left side surface as viewed from the front surface of the main body 1 of the portable electrocardiographic device 100, a power switch 7, a power LED 8, a BLE communication button 9, a communication LED 10, a residual memory display LED 11, and a battery replacement LED 12 are disposed vertically side by side. The power switch 7 is a depression switch configured to turn on the power of the portable electrocardiographic device 100. The power LED 8 is a light-emitting element that is turned on when the power is turned on. The BLE communication button 9 is an operation part configured to cause communication with Bluetooth (trade name) Low Energy (BLE) scheme-compliant apparatuses to function. The communication LED 10 is a light-emitting element that is turned on during communication. Note that the communication function that the portable electrocardiographic device 100 has is not limited to that of the BLE-scheme, and may be of a wireless communication method such as infrared communications or information transmission via ultrasonic waves, or of a wired communication scheme in which connections are established via cables, connectors, and the like. The residual memory display LED 11 is a light-emitting element that indicates the state of the remaining capacity of a memory unit to be described later. The battery replacement LED 12 is a light-emitting element that is turned on to prompt for battery replacement when the power of the power source (battery) included in the portable electrocardiographic device 100 falls below a predetermined value.

A lead type setting input unit 13 and a lead type display LED 14 are disposed on the right side surface as viewed from the front surface of the main body 1 of the portable electrocardiographic device 100. The lead type display LED 14 displays, among the plurality of lead systems, which lead system is used to detect the electrocardiographic waveform. The lead type display LED 14 includes a display LED 14a for lead I, a display LED 14b for lead II, a display LED 14c for lead III, a display LED 14d for lead V1, a display LED 14e for lead V2, a display LED 14f for lead V3, a display LED 14g for lead V4, a display LED 14h for lead V5, and a display LED 14i for lead V6. On the right side surface of the main body 1, indications indicating the respective lead systems are provided in the vicinity of the display LEDs 14a to 14i. The lead type setting input unit 13 is a button that is depressed to switch among the lead types. For example, when the power of the portable electrocardiographic device 100 is turned on, lead I is set as the initial setting, and the display LED 14a for lead I is turned on. However, selecting and depressing the button of the lead type setting input unit 13 causes lead II to be set and the display LED 14b for lead II to be turned on. Similarly, each time the button of the lead type setting input unit 13 is depressed, the set lead type sequentially switches among lead III, lead V1, lead V2, lead V3, lead V4, lead V5, and lead V6, and the corresponding lead type display LEDs 14c to 14i are sequentially turned on. The lead type display LED 14 and the lead type setting input unit 13 correspond to the setting unit of the present invention.

The lead type display LED is not limited to the one in which an LED is provided for each lead type as described above, and may be one in which one LED is provided that emits light in a different color for each of the lead types and in which the lead type is distinguished by the light emission color of the LED.

Furthermore, a removable battery cover 15 is provided at the rear surface of the main body 1 of the portable electrocardiographic device 100.

Here, for example, when measurement with lead I is performed in electrocardiographic measurement, with the portable electrocardiographic device 100 held by the right hand, the first electrode 2 provided at the bottom of the main body 1 is brought into contact with the left palm. When holding the portable electrocardiographic device 100, the tip of the right hand index finger is brought into contact with the second electrode 3, and the middle phalanx of the right hand index finger is brought into contact with the third electrode 4. For example, the subject performs electrocardiographic measurement while pushing the first electrode 2 provided at the bottom, from the top side of the main body 1 at which the second electrode 3 and the third electrode 4 are provided, in the pressing direction that is the direction to the left palm. Here, the tip and the middle phalanx of the right hand index finger and the left palm correspond to the predetermined location of the subject's body in the present invention.

When measurement with lead II is performed in electrocardiographic measurement, with the portable electrocardiographic device 100 held by the right hand, the first electrode 2 provided at the bottom of the main body 1 is brought into contact with the left upper thigh (or left ankle). When holding the portable electrocardiographic device 100, the tip of the right hand index finger is brought into contact with the second electrode 3, and the middle phalanx of the right hand index finger is brought into contact with the third electrode 4. Here, the tip and the middle phalanx of the right hand index finger and the left upper thigh (or left ankle) correspond to the predetermined location of the subject's body in the present invention.

Furthermore, when measurement with lead III is performed in electrocardiographic measurement, with the portable electrocardiographic device 100 held by the left hand, the first electrode 2 provided at the bottom of the main body 1 is brought into contact with the left upper thigh (or left ankle). When holding the portable electrocardiographic device 100 by the left hand, the tip of the left hand index finger is brought into contact with the third electrode 4, and the middle phalanx of the left hand index finger is brought into contact with the second electrode 3. For example, the subject performs electrocardiographic measurement while pushing the first electrode 2 provided at the bottom, from the top side of the main body 1 at which the second electrode 3 and the third electrode 4 are provided, in the pressing direction that is the direction to the left upper thigh (or left ankle). Here, the tip and the middle phalanx of the left hand index finger and the left upper thigh (or left ankle) correspond to the predetermined location of the subject's body in the present invention.

Furthermore, when measurement with lead V4 is performed in electrocardiographic measurement, with the portable electrocardiographic device 100 held by the right hand, the subject brings the first electrode 2 provided at the bottom of the main body 1 into contact with the skin of the left chest slightly to the left of the epigastric region and on the lower side of the left nipple. When holding the portable electrocardiographic device 100, the right hand index finger is brought into contact with the second electrode 3, and the middle phalanx of the right hand index finger is brought into contact with the third electrode 4. Then, the electrocardiographic measurement is performed while the first electrode 2 provided at the bottom is being pushed, from the top side of the main body 1 at which the second electrode 3 and the third electrode 4 are provided, in the pressing direction that is the direction to the measurement site. Here, the tip and the middle phalanx of the right hand index finger and the skin of the left chest slightly to the left of the epigastric region and on the lower side of the left nipple correspond to the predetermined location of the subject's body in the present invention.

### Configuration of Portable Electrocardiographic Device

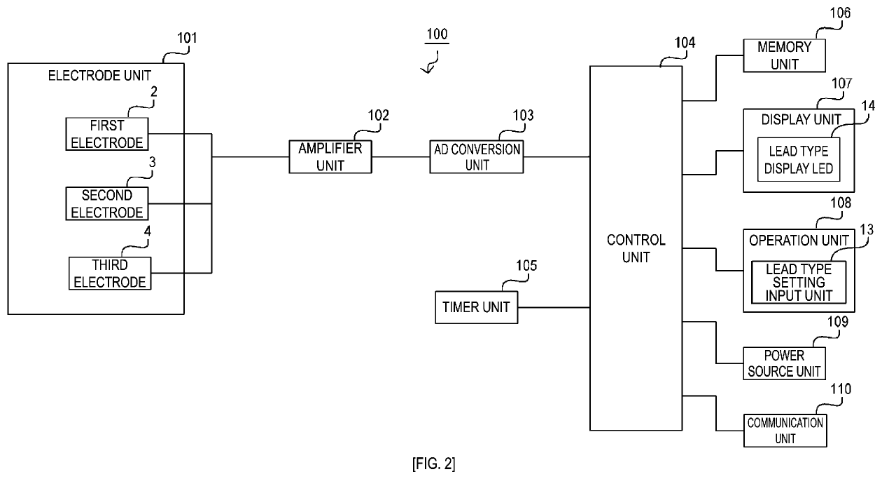

Next, the configuration of the portable electrocardiographic device 100 will be described. FIG. 2 is a functional block diagram illustrating an example of a configuration of the portable electrocardiographic device 100 according to the present embodiment.

As illustrated in FIG. 2, the portable electrocardiographic device 100 includes an electrode unit 101, an amplifier unit 102, an analog-to-digital (AD) conversion unit 103, a control unit 104, and a timer unit 105. The configuration of the portable electrocardiographic device 100 also includes a memory unit 106, a display unit 107, an operation unit 108, a power source unit 109, and a communication unit 110. The control unit 104, the timer unit 105, the memory unit 106, the display unit 107, the operation unit 108, the power source unit 109, and the communication unit 110 are connected to each other.

The electrode unit 101 includes the first electrode 2 and the third electrode 4 that function as a pair of measurement electrodes, and the second electrode 3 that functions as ground (GND) electrode. Through the electrode unit 101 brought into contact with the skin of the subject, an electrocardiographic waveform in a predetermined period is detected. The electrocardiographic waveforms detected by each of the electrodes of the electrode unit 101 are each input to the amplifier unit 102 connected to the electrode unit. The amplifier unit 102 amplifies a signal detected by the electrode unit 101, and outputs the resultant signal to the AD conversion unit 103. The AD conversion unit 103 performs digital conversion on the detection signal of the electrocardiographic waveform amplified by the amplifier unit 102, and outputs the resultant signal to the control unit 104.

The control unit 104 is a processor such as a central processing unit (CPU) that controls the portable electrocardiographic device 100. The control unit 104 executes a program stored in the memory unit 106, whereby various processing is executed, such as setting of the lead type, and electrocardiographic waveform measurement and analysis in accordance with the lead system. Here, the control unit 104, which executes analysis processing of electrocardiographic waveforms in accordance with the lead system, corresponds to the analysis unit of the present invention.

The timer unit 105 is a unit to receive instructions from the control unit 104, and count various time or periods related to electrocardiographic waveform measurement.

The memory unit 106 is configured by including a main storage device such as a read-only memory (ROM) and a random access memory (RAM), and also a long-term storage medium such as flash memory, for example. The memory unit 106 stores various programs related to electrocardiographic waveform measurement and analysis, and various information for detecting abnormal waveforms and the like. Here, the memory unit 106 corresponds to the storage unit of the present invention.

The display unit 107 is a unit to display various information related to electrocardiographic waveform measurement. The display unit 107 includes the measurement notification LED 5, the abnormal waveform detection LED 6, the power LED 8, the communication LED 10, the residual memory display LED 11, the battery replacement LED 12, and the lead type display LED 14. The display unit 107 may include a unit to display various information by an image and/or video, such as a liquid crystal display.

The operation unit 108 is a unit to receive operation inputs from the subject or the user. The operation unit 108 includes the power switch 7, the BLE communication button 9, and the lead type setting input unit 13. The power source unit 109 is a unit to supply power for causing the portable electrocardiographic device 100 to function, and includes a battery, a secondary battery, or the like. The communication unit 110 is a communication interface that controls signal transmission and reception to and from an apparatus such as a smartphone 200. The communication function provided by the communication unit 110 may be BLE communications, for example, but other known wireless and wired communication schemes can be employed.

### Smartphone

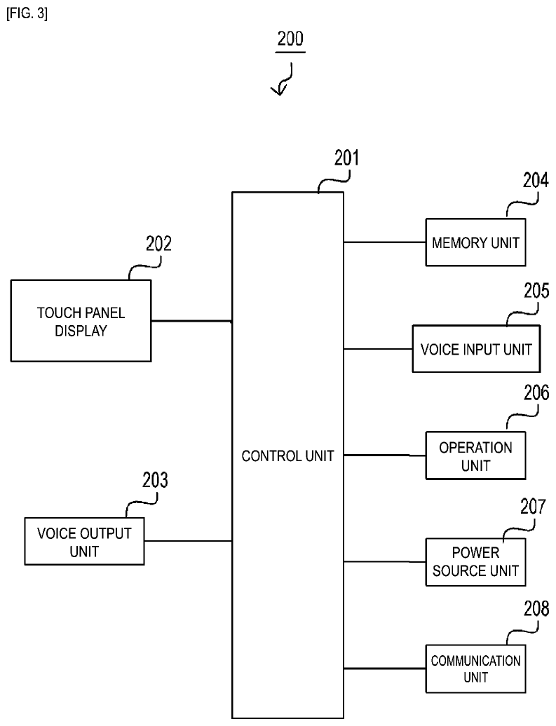

FIG. 3 is a block diagram illustrating a configuration of the smartphone 200. As will be described later, the smartphone 200 constitutes an electrocardiographic measurement system in cooperation with the portable electrocardiographic device 100. The smartphone 200 includes a control unit 201, a touch panel display 202, a voice output unit 203 such as a speaker, a memory unit 204, a voice input unit 205 such as a microphone, an operation unit 206 such as a button, a power source unit 207, and a communication unit 208, which is a communication interface that controls signal transmission and reception to and from the portable electrocardiographic device 100 by a scheme such as BLE communication. Executing a program stored in the memory unit 204 in the control unit 201 causes various processing to be executed, such as setting of the lead type, and displaying and storing of electrocardiographic waveforms and analysis results. For the smartphone 200, which is an example of a portable communication terminal provided communicably with the portable electrocardiographic device 100, a known configuration can be employed, and thus no detailed description will be given. Here, the memory unit 204 of the smartphone 200 corresponds to the storage unit of the present invention.

### Basic Electrocardiographic Measurement Processing

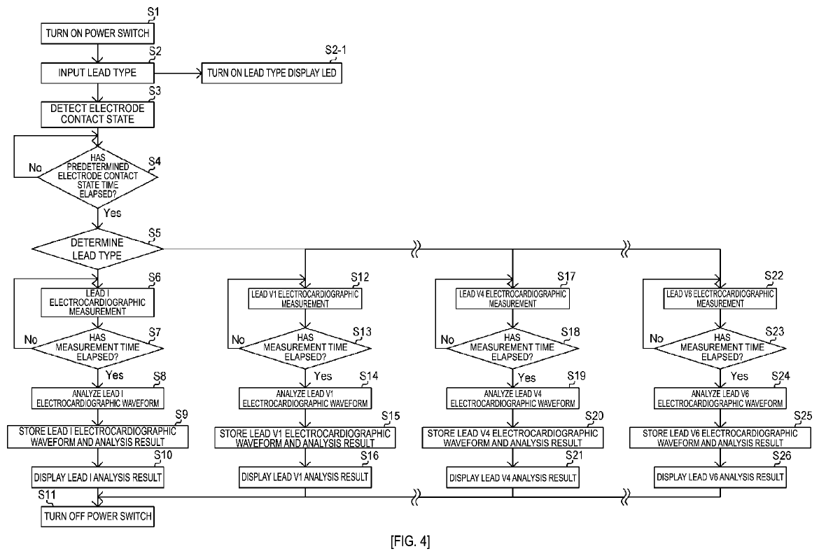

FIG. 4 is a flowchart illustrating, of the electrocardiographic measurement processing using the portable electrocardiographic device 100, procedures for basic electrocardiographic waveform measurement processing.

First, the power switch 7 of the portable electrocardiographic device 100 is depressed to turn on the power (step S1). At this time, the power LED 8 is turned on to indicate that the power is on.

Next, the subject or the user inputs, through the lead type setting input unit 13, the lead type with which the measurement is to be performed (step S2). For example, when the subject is to measure the electrocardiographic waveform with lead V5, from the state in which the display LED 14a for lead I is turned on by the initial setting, the button of the lead type setting input unit 13 is depressed six times. This sequentially switches the lead type to II, III, and the like until the display LED 14g for lead V4 is turned on, indicating that electrocardiographic measurement with lead V4 is set (step S2-1).

In lead V4, the tip of the right hand index finger is brought into contact with the second electrode 3, and the middle phalanx of the right hand index finger is brought into contact with the third electrode 4. Then, the first electrode 2 is brought into contact with the skin of the left chest slightly to the left of the epigastric region and on the lower side of the left nipple. Electrical signals respectively acquired via the electrodes 2, 3, and 4 are amplified in the amplifier unit 102 and digitally converted in the AD conversion unit 103 to generate a contact state detection signal. The contact state detection signal generated in this way is transmitted to the control unit 104, and the contact state between the subject and each of the electrodes 2, 3, and 4 is detected (step S3).

The control unit 104 determines whether a predetermined time has elapsed with the electrode contact state being maintained (step S4). If a “NO” determination is made in step S4, step S4 is repeated. If a “YES” determination is made in step S4, the control unit 104 determines the lead type (step S5).

When lead V4 is set in step S2, the control unit 104 determines that the lead type is lead V4 in step S5, and proceeds to step S17 to start electrocardiographic waveform measurement with lead V4.

The control unit 104 causes the time elapsed since the start of measurement to be counted in the timer unit 105, and determines whether a predetermined measurement time has elapsed (step S18).

In the case of “NO” in step S18, the processing returns to step S17 to continue electrocardiographic waveform measurement.
In the case of “YES” in step S18, the control unit 104 analyzes the electrocardiographic waveform with lead V4 (step S19). Upon completion of electrocardiographic waveform analysis, the measurement notification LED 5 is turned on to notify the subject of measurement completion.
Since the characteristics of the identifying parameters for the electrocardiographic waveform vary depending on the lead system, it is desirable that a lead system be set with which electrocardiographic waveform data suited to the information desired to be acquired can be obtained. Furthermore, in electrocardiographic waveform data analysis, analyzing the electrocardiographic waveform in accordance with the lead system allows optimal electrocardiographic waveform analysis.

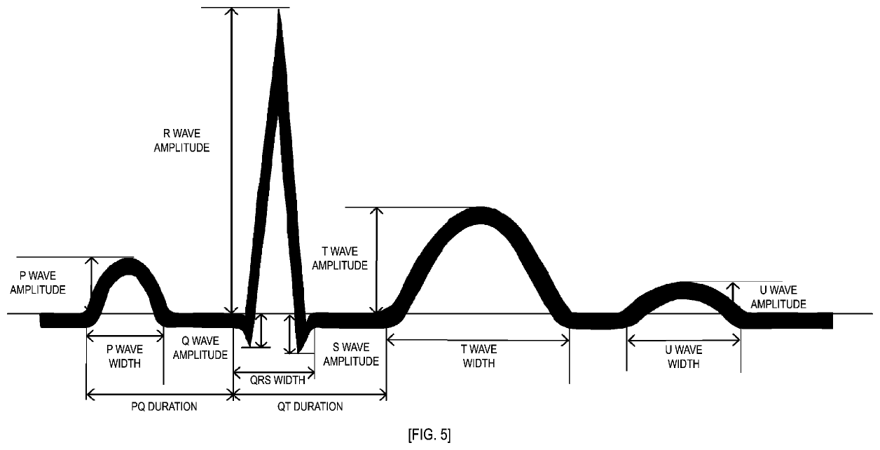

FIG. 5 illustrates typical electrocardiographic waveform parameters. P-wave amplitude and P-wave width are defined for P waves. Q-wave amplitude is defined for Q waves. PQ duration is defined for P waves and Q waves. R-wave amplitude is defined for R waves. S-wave amplitude is defined for S waves. QRS width is defined for Q waves, R waves, and S waves. T-wave amplitude and T-wave width are defined for T waves. QT duration is defined for Q waves and T waves. U-wave amplitude and U-wave width are defined for U waves. One or a plurality of numerical values of these portions of the electrocardiogram or a value calculated based on the one or the plurality of numerical values can be used as an identifying parameter or parameters for the waveform of the electrocardiogram.

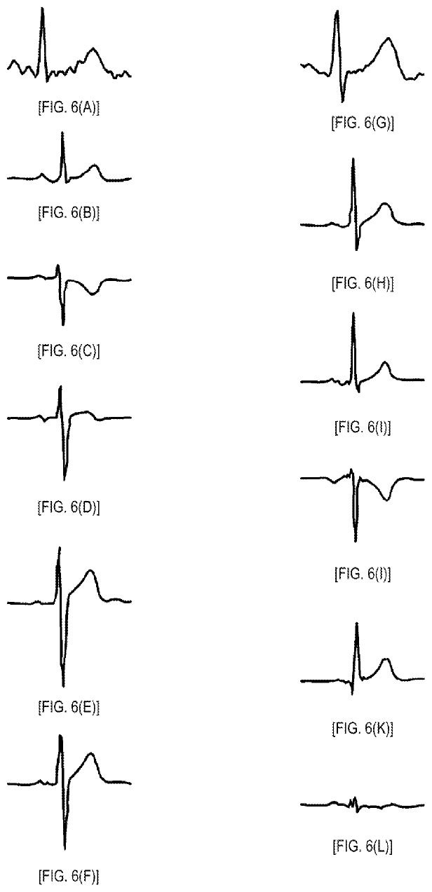

FIG. 6 illustrates typical electrocardiographic waveforms for lead types. FIG. 6(A) is an electrocardiographic waveform measured with lead I. FIG. 6(B) is an electrocardiographic waveform measured with lead II. FIG. 6(C) is an electrocardiographic waveform measured with lead III. FIG. 6(D) is an electrocardiographic waveform measured with lead V1. FIG. 6(E) is an electrocardiographic waveform measured with lead V2. FIG. 6(F) is an electrocardiographic waveform measured with lead V3. FIG. 6(G) is an electrocardiographic waveform measured with lead V4. FIG. 6(H) is an electrocardiographic waveform measured with lead V5. FIG. 6(I) is an electrocardiographic waveform measured with lead V6. FIG. 6(J) is an electrocardiographic waveform measured with lead aVR. FIG. 6(K) is an electrocardiographic waveform measured with lead aVL. FIG. 6(L) is an electrocardiographic waveform measured with lead aVF.

As illustrated in FIG. 6(A), in the electrocardiographic waveform with lead I, whether there is an irregular pulse wave can be approximately determined by the interval between R waves, which have high peak values. However, peak values are small in the electrocardiographic waveform with lead I, and thus P waves and F waves (irregular baseline fluctuations) are easily buried in noise. Accordingly, to measure typical electrocardiographic waveform parameters as illustrated in FIG. 5, collecting electrocardiographic waveform data with a lead system in which the PQRST shapes are large, such as lead V4, allows more optimal electrocardiographic measurement. Furthermore, as an example of electrocardiographic waveform analysis in accordance with the lead system, with lead V4, ST changes are easily captured, and thus ST elevation may also be determined with lead systems other than lead V4, ST changes are difficult to capture, and thus any other determination may be performed without ST elevation being determined. However, the present invention is not limited to this example.

Upon completion of electrocardiographic waveform analysis, the control unit 104 stores the electrocardiographic waveform with lead V4 and the analysis result in association with each other in a predetermined region of the memory unit 106 (step S20).

Then, the control unit 104 displays the result of electrocardiographic waveform analysis (step S21). Specifically, when an abnormal waveform is detected as a result of electrocardiographic waveform analysis, the abnormal waveform detection LED 6 is turned on to notify the subject that an abnormal waveform is detected.
After the analysis result of the electrocardiographic waveform is displayed and the electrocardiographic measurement processing is completed, the subject or the user depresses the power switch 7 again to turn off the power. The power may be caused to be turned off when a predetermined time has elapsed since the analysis result of the electrocardiographic waveform is displayed without any operation on the power switch 7.

In the example described above, a case in which lead V4 is set as the lead type in step S2 has been described. However, even when lead I is set as the lead type in step S2, the control unit 104 executes the processing in the same or similar procedures. In other words, an electrocardiographic waveform is measured with lead I (step S6), the elapse of a predetermined measurement time is waited for (step S7), the electrocardiographic waveform with lead I is analyzed (step S8), and the electrocardiographic waveform with lead I and the analysis result are stored in a predetermined region of the memory unit 106 (step S9). Then, when an abnormality is detected in the electrocardiographic waveform, the abnormal waveform detection LED 6 is turned on and the analysis result is displayed (step S10) and then the electrocardiographic measurement processing is terminated. Depressing the power switch 7 turns off the power (step S11).

FIG. 4 also describes the processing in a case in which lead V1 is set in step S2 (step S12 to step S16), and the processing in a case in which lead V6 is set in step S2 (step S22 to step S26), rather than lead I and lead V4. However, such processing is the same as or similar to the processing described for lead I and lead V4, and thus description thereof is omitted. The processing for the other lead types of which description is omitted in FIG. 4, that is, lead V2, lead V3, and lead V5, is also the same as or similar to the processing described for lead I and lead V4, and thus description thereof is omitted.

### Processing for Adding Electrocardiographic Waveform Remeasurement in Different Lead System

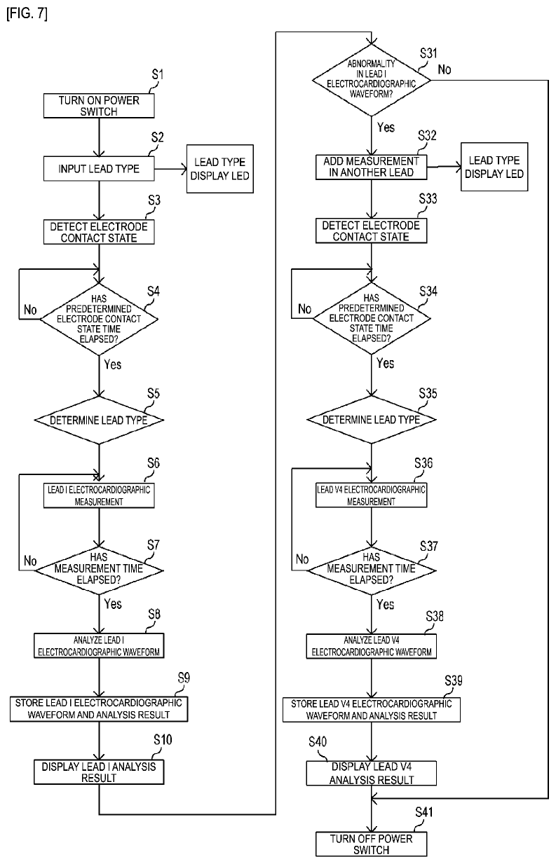

Hereinafter, of the electrocardiographic waveform measurement processing using the portable electrocardiographic device 100, the processing for adding electrocardiographic waveform remeasurement in a different lead system will be described with reference to FIG. 7. The same reference numerals are used for procedures that are the same as or similar to those of the electrocardiographic waveform measurement processing illustrated in FIG. 4, and detailed description thereof is omitted.

Step S1 to step S10 are the same as or similar to the electrocardiographic waveform measurement processing illustrated in FIG. 4. Here, an example in which lead I is set as the lead type in step S2 will be described.

Electrocardiographic waveform measurement with lead I is performed in step S6 and step S7. The electrocardiographic waveform is analyzed in step S8. Then, in step S9, the electrocardiographic waveform with lead I and the analysis result are stored in a predetermined region of the memory unit 106. If there is an abnormality in the electrocardiographic waveform with lead I in step S8, the abnormal waveform detection LED 6 is turned on in step S10. If there is no abnormality in the electrocardiographic waveform with lead I in step S8, the measurement analysis result is displayed in step S10. That there is no abnormality in the analysis result is indicated by not turning on the abnormal waveform detection LED 6, or turning on or causing to blink the abnormal waveform detection LED 6 in a manner different from when there is an abnormality in the analysis result. Here, the abnormal waveform detection LED 6 corresponds to the display unit of the present invention.

Here, in the next step S31, the control unit 104 determines, as a result of the electrocardiographic waveform analysis, whether there is an abnormality in the electrocardiographic waveform with lead I. Here, as a result of the electrocardiographic waveform analysis, whether there is an abnormality in the electrocardiographic waveform is determined by whether the analysis result of the electrocardiographic waveform satisfies a predetermined condition. Examples of predetermined conditions include conditions such as that arrhythmia is observed, that atrial fibrillation is observed, and that a defect in waveform quality is observed. When any of such conditions is satisfied, the control unit 104 determines that there is an abnormality in the electrocardiographic waveform.

If a “NO” determination is made in step S31, the electrocardiographic measurement process is terminated. Depressing the power switch 7 turns off the power (step S41). If a “YES” determination is made in step S31, the processing proceeds to step S32.

In lead I, whether there is an irregular pulse wave can be approximately determined by the interval between R waves, which have high peak values (see FIG. 6(A)). However, in lead I, the sizes of PQRST waves, which are typical electrocardiographic waveform parameters illustrated in FIG. 5, are small, and thus the optimal analysis is difficult. Therefore, terminating electrocardiographic waveform measurement at this stage means ending with a simplified electrocardiographic waveform measurement with lead I without enabling more accurate electrocardiographic waveform measurement and analysis. Accordingly, in electrocardiographic waveform measurement with lead I, when an abnormal electrocardiographic waveform such as arrhythmia is detected or when the waveform quality is defective, to prompt for electrocardiographic waveform measurement with another lead system, the control unit 104 causes to blink the lead type display LED 14 corresponding to the lead system in which electrocardiographic waveform measurement is to be additionally performed (step S32). As described above, the control unit 104 corresponds to the remeasurement facilitating unit of the present invention, which determines whether there is an abnormality in the electrocardiographic waveform and, when it is determined that there is an abnormality, performs the processing for causing to blink the lead type display LED 14 for prompting for remeasurement.

For example, when it is desired to more precisely grasp and analyze the electrocardiographic waveform pattern, electrocardiographic waveform remeasurement with lead V4 is added and the corresponding display lead 14g is caused to blink. In this way, the processing in step S33 to step S35 after remeasurement with lead V4 is set is the same as or similar to that in step S3 to step S5 in FIG. 4, and the processing in step S36 to step S41 is the same as or similar to that in step S17 to step S21 and step S11 in FIG. 4.

The lead system added to the electrocardiographic waveform measurement with lead I is not limited to lead V4 described above, and various lead systems can be set. For example, when the control unit 104 determines, as a result of electrocardiographic waveform analysis in step S8, that there is a possibility of atrial fibrillation (AF), it is difficult to make a more reliable determination on atrial fibrillation with lead I, and it is preferable to check the presence or absence of P waves or F waves (irregular baseline fluctuations). In this case, electrocardiographic waveform remeasurement with lead V1 is added in step S32. The electrocardiographic waveform with lead V1 is a waveform illustrated by FIG. 6(D). Thus, additionally performing remeasurement with lead V1, in which P waves and F waves are easily grasped, allows electrocardiographic waveform data to be collected that is more beneficial in determining the presence or absence of atrial fibrillation.

### Basic Electrocardiographic Measurement Processing in Which Portable Electrocardiographic Device and Smartphone Cooperate with Each Other

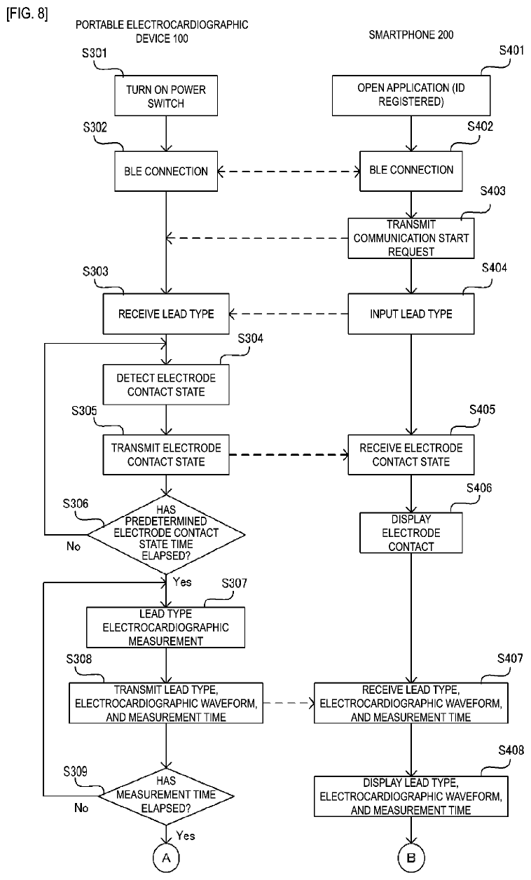

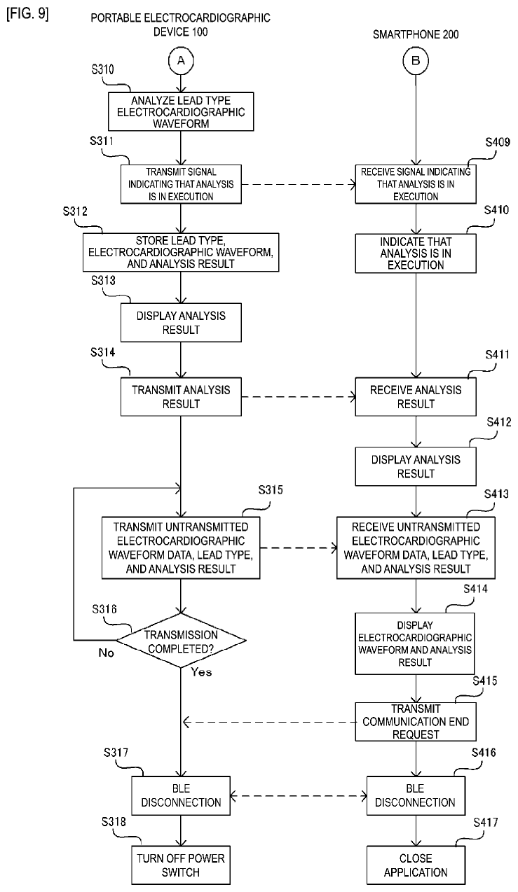

FIG. 8 and FIG. 9 are each a flowchart illustrating procedures in which, while engaging in BLE communication with each other, the portable electrocardiographic device 100 and a terminal equipped with a BLE-scheme communication function such as the smartphone 200 measure a basic electrocardiographic waveform. FIG. 8 and FIG. 9 each illustrate a series of procedures.

First, the power switch 7 of the portable electrocardiographic device 100 is depressed to turn on the power (step S301). On the other hand, in the smartphone 200, an application for electrocardiographic measurement is opened (step S401). The description herein assumes that registration of the ID of the subject and the like has been completed at the time of the initial setting described above.

Next, a BLE connection is established between the portable electrocardiographic device 100 and the smartphone 200 in accordance with a predetermined procedure (step S302 and step S402).

Once a BLE connection is established between the portable electrocardiographic device 100 and the smartphone 200, the smartphone 200 transmits a communication start request to the portable electrocardiographic device 100 (step S403).

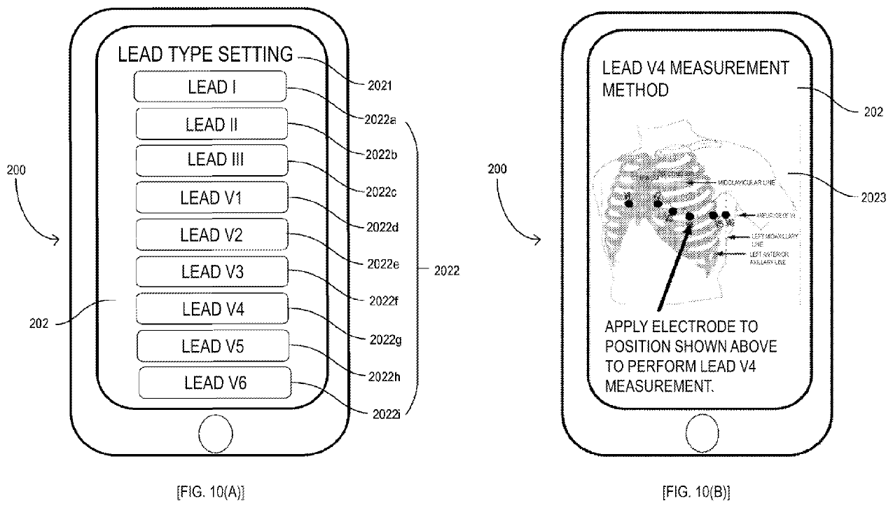

Next, in the smartphone 200, the control unit 201 receives the input of the lead type (step S404). FIG. 10(A) is a display example of the touch panel display 202 when the subject or the user inputs the lead type setting in the smartphone 200. The touch panel display 202 displays, on a lead type setting screen 2021 together with characters, buttons 2022 for selecting the lead system to be set from among a plurality of lead systems. The buttons 2022 for selecting the lead type include buttons each corresponding to the corresponding lead system of the plurality of lead systems. In other words, the buttons 2022 include a button 2022a for setting lead I, a button 2022b for setting lead II, a button 2022c for setting lead III, a button 2022d for setting lead V1, a button 2022e for setting lead V2, a button 2022f for setting lead V3, a button 2022g for setting lead V4, a button 2022h for setting lead V5, and a button 2022i for setting lead V6. Each of the buttons 2022a to 2022i comes with an indication associated with the corresponding lead system. For example, when selecting electrocardiographic measurement with lead V4, the subject or the user touches the button 2022g on the touch panel display 202. Once lead V4 is set, the touch panel display 202 displays a guide screen 2023 that describes, using a figure and characters, the position (measurement site) with which the subject is to bring the electrode 2 of the portable electrocardiographic device 100 into contact in accordance with the set lead system, as illustrated in FIG. 10(B). Here, a guide screen corresponding to lead V4 is illustrated. However, the same or similar guide screens can be displayed for the lead systems selectable by the subject or the user. Displaying the measurement site with which the electrode 2 is to be brought into contact in accordance with the set lead type on the touch panel display 202 of the smartphone 200 allows the subject to bring the electrode 2 into contact with the correct position. Guiding the subject to the measurement site by such a guide screen 2023 allows the optimal lead to be more reliably set, and the electrocardiographic waveform to be correctly measured. Here, the buttons 2022 including the buttons 2022a to 2022i correspond to the setting unit of the present invention.

The lead type set in step 5404 is transmitted from the smartphone 200 to the portable electrocardiographic device 100. The portable electrocardiographic device 100 receives the lead type (step S303), and stores the same in a predetermined region of the memory unit 106.

Next, in the portable electrocardiographic device 100, the control unit 104 detects the electrode contact state (step S304). Specifically, when measurement with lead V4 is performed with the portable electrocardiographic device 100, the tip of the right hand index finger is brought into contact with the second electrode 3, and the middle phalanx of the right hand index finger is brought into contact with the third electrode 4. Then, the first electrode 2 is brought into contact with the skin of the left chest slightly to the left of the epigastric region and on the lower side of the left nipple. Furthermore, when measurement with lead I is performed with the portable electrocardiographic device 100, the tip of the right hand index finger is brought into contact with the second electrode 3, and the middle phalanx of the right hand index finger is brought into contact with the third electrode 4. Then, the left palm is brought into contact with the first electrode 2. As described above, the subject respectively brings the electrodes 2, 3, and 4 into contact with the measurement sites in accordance with the set lead type. Electrical signals respectively acquired via the electrodes 2, 3, and 4 are amplified in the amplifier unit 102 and digitally converted in the AD conversion unit 103 to generate a contact state detection signal. The contact state detection signal generated in this way is transmitted to the control unit 104, and the contact state between the subject and each of the electrodes 2, 3, and 4 is detected.

In the portable electrocardiographic device 100, information indicating the electrode contact state is transmitted to the smartphone 200 (step S305). Upon receiving the information indicating the electrode contact state (step S405), the smartphone 200 displays the electrode contact state on the touch panel display 202 and the like (step S406) to notify the subject that normal contact is maintained with each of the electrodes 2, 3, and 4.

The control unit 104 determines whether a predetermined time has elapsed with the electrode contact state being maintained (step S306).

If a “NO” determination is made in step S306, the processing returns to step S304.
If a “YES” determination is made in step S306, the control unit 104 starts electrocardiographic measurement in accordance with the set lead type (step S307).

Once electrocardiographic measurement is started, the portable electrocardiographic device 100 performs streaming communication to and from the smartphone 200, and transmits lead type information, electrocardiographic waveform information, and measurement time information to the smartphone 200 (step S308). The measurement time information is information related to the time elapsed since the start of electrocardiographic measurement, which is counted in the timer unit 105. Here, the measurement time information is information indicating the remaining measurement time obtained by subtracting, from a predetermined time, the time elapsed since the start of electrocardiographic measurement. The information on the time elapsed since the start of electrocardiographic measurement may be transmitted from the portable electrocardiographic device 100 to the smartphone 200, and the processing of subtracting the elapsed time from the predetermined time may be performed on the smartphone 200 side. On the other hand, the smartphone 200 receives the lead type information, the electrocardiographic waveform information, and the measurement time information from the portable electrocardiographic device 100 (step S407).

The smartphone 200 displays the lead type, the electrocardiographic waveform, and the measurement time on the touch panel display 202 (step S408). In this way, the subject is notified of the lead type, that the electrocardiographic measurement is being normally performed, and the remaining measurement time.

The lead type displayed on the touch panel display 202 can be utilized to instruct the subject on the proper measurement posture. Furthermore, when a lead type different from the lead system intended by the subject is displayed on the touch panel display 202, a prompt for remeasurement in the proper measurement posture can be performed.

Whether a predetermined measurement time (e.g., 30 seconds) has elapsed since the start of electrocardiographic waveform measurement is determined (step S309).

If a “NO” determination is made in step S309, the processing returns to step S307 to continue electrocardiographic measurement.
If a “YES” determination is made in step S309, the control unit 104 analyzes the electrocardiographic waveform in accordance with the set predetermined lead system (step S310). Analyzing the electrocardiographic waveform in accordance with the set predetermined lead system allows accurate analysis.

During electrocardiographic waveform analysis, the control unit 104 transmits information indicating that electrocardiographic waveform analysis is in execution to the smartphone 200 (step S311). Upon receiving the information indicating that electrocardiographic waveform analysis is in execution from the portable electrocardiographic device 100 (step S409), the smartphone 200 displays information indicating that electrocardiographic waveform analysis is in execution on the touch panel display 202 (step S410).

Upon completion of electrocardiographic waveform analysis, the control unit 104 stores the lead type, the electrocardiographic waveform, and the analysis result in association with one another in a predetermined region of the memory unit 106 (step S312). Storing the lead type in association with the electrocardiographic waveform and the analysis result in the predetermined region of the memory unit 106 allows useful information to be provided when the physician reads out the electrocardiographic waveform and utilizes the same for diagnosis or the like. The lead type, the electrocardiographic waveform, and the analysis result associated with one another may be stored only on the smartphone 200 side without being stored in the memory unit 106 of the portable electrocardiographic device 100. Furthermore, only one of the lead type, the electrocardiographic waveform, and the analysis result may be stored in the memory unit 106 of the portable electrocardiographic device 100. When an abnormal waveform is detected by electrocardiographic waveform analysis, the control unit 104 may cause the abnormal waveform detection LED 13 to blink to notify the subject of the abnormal waveform detection.

Furthermore, upon completion of electrocardiographic waveform analysis, the control unit 104 transmits the analysis result to the smartphone 200 by high-speed data communication (step S314). At this time, the smartphone 200 receives the analysis result transmitted from the portable electrocardiographic device 100 (step S411), and displays the analysis result, that is, whether the electrocardiographic measurement result is normal and without any problems or whether an abnormal waveform has been detected, on the touch panel display 202 (step S412).

Then, if there is any electrocardiographic waveform data, lead type determination result data, or analysis result that has not yet been transmitted to the portable electrocardiographic device 100, the control unit 104 transmits such information to the smartphone 200 in the descending chronological order by high-speed data communication (step S315). At this time, the smartphone 200 receives the untransmitted electrocardiographic waveform data, lead type data, and analysis result from the portable electrocardiographic device 100 (step S413), and stores the same in a predetermined region of the memory unit 204. Then, the smartphone 200 displays the analysis result, such as whether the latest electrocardiographic waveform and electrocardiographic measurement result are normal or whether an abnormal waveform has been detected, on the touch panel display 202 (step S414).

Upon completion of transmission of the untransmitted electrocardiographic waveform data, lead type determination result data, and analysis result (step S316), in response to a communication end request transmitted from the smartphone 200 (step S415), the portable electrocardiographic device 100 disconnects the BLE communication (step S317). In response to the disconnection of the BLE communication in the portable electrocardiographic device 100, the BLE communication is also disconnected on the smartphone 200 side (step S416).

After the BLE communication is disconnected, the power switch 7 is turned off in the portable electrocardiographic device 100 (step S318). The control unit 104 may automatically turn off the power switch 7 when a predetermined time has elapsed since BLE disconnection, or the subject or the user may depress the power switch 7 to turn off the same. On the other hand, in the smartphone 200, the application is closed after the BLE communication is disconnected (step S417). In this way, electrocardiographic measurement in the portable electrocardiographic device 100 in cooperation with the smartphone 200 is completed.

### Processing in which Portable Electrocardiographic Device and Smartphone Cooperate with Each Other to Add Electrocardiographic Waveform Remeasurement in Different Lead System

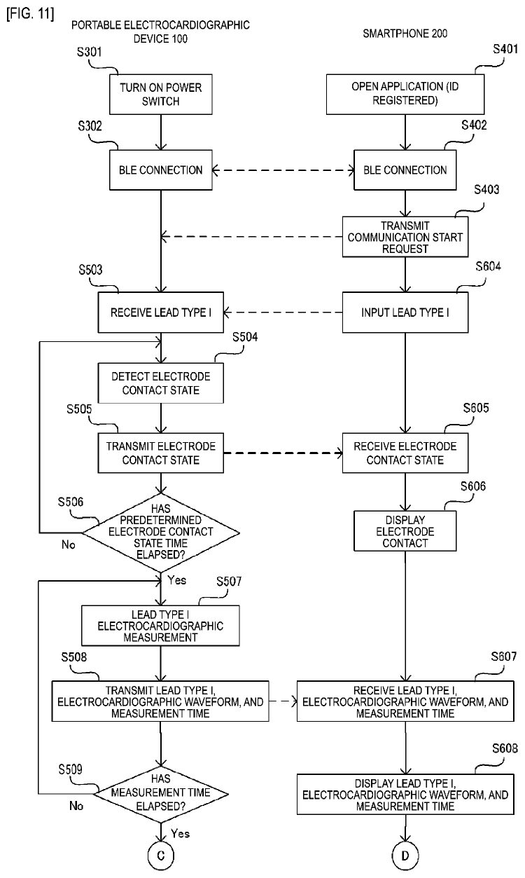

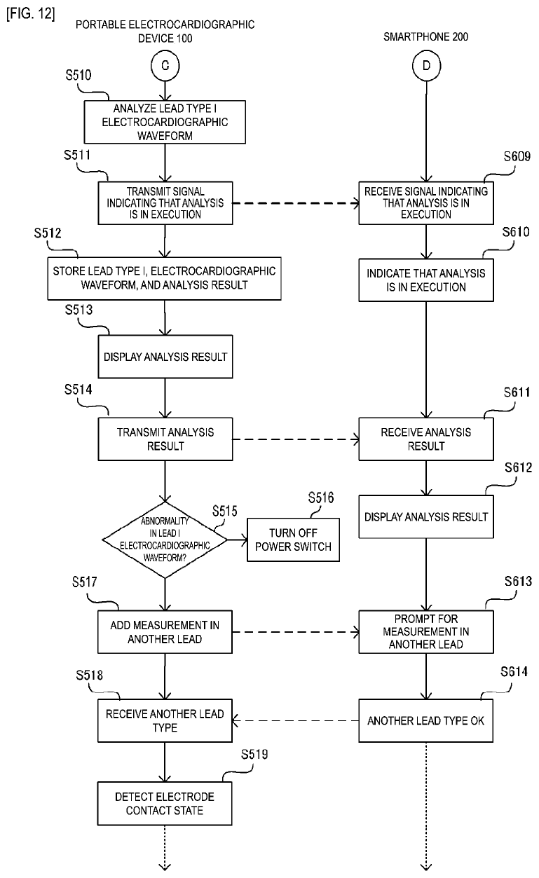

FIG. 11 and FIG. 12 are each a flowchart illustrating procedures in which, while engaging in BLE communication with each other, the portable electrocardiographic device 100 and a terminal equipped with a BLE-scheme communication function such as the smartphone 200 measure an electrocardiographic waveform in one lead system, and then measure an electrocardiographic waveform in a different lead system. FIG. 11 and FIG. 12 each illustrate a series of procedures. The same reference numerals are used for processing that is common to the basic electrocardiographic waveform measurement processing illustrated in FIG. 8 and FIG. 9, and detailed description thereof is omitted.

First, the processing of step S301 and step S302 in the portable electrocardiographic device 100 and step S401 to step S403 in the smartphone 200 is the same as or similar to that illustrated in FIG. 8, and thus description thereof is omitted.

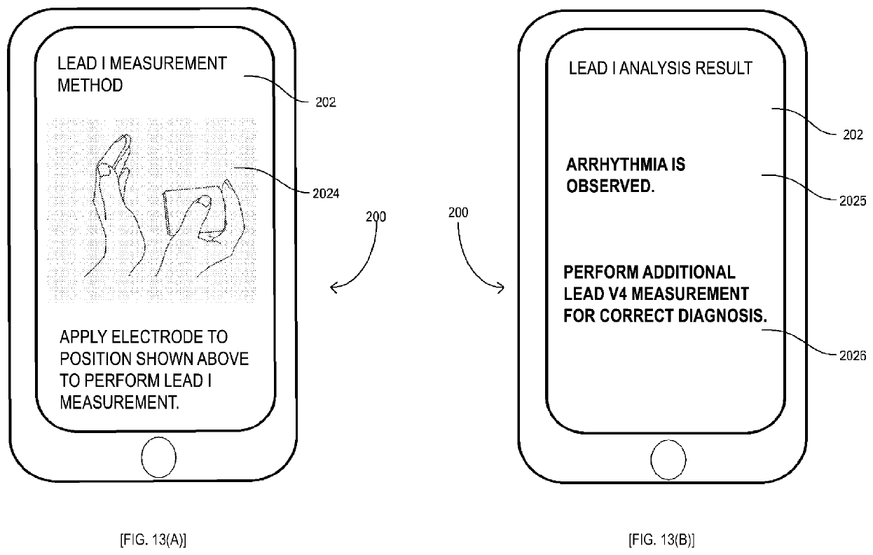

Subsequently, in the smartphone 200, the control unit 201 receives the input of the lead type (step S604). At this time, lead I is selected and input on the smartphone 200. At this time, the subject or the user touches the button 2022a for setting lead I on the touch panel display 202 of the smartphone 200. Once lead I is set, the touch panel display 202 displays a guide screen 2024 that describes, using a figure and characters, the position (measurement site) with which the subject is to bring the electrode 2 of the portable electrocardiographic device 100 into contact in accordance with the set lead I, as illustrated in FIG. 13(A). Here, a guide screen corresponding to lead I is illustrated. However, the same or similar guide screens can be displayed for the lead systems selectable by the subject or the user. Displaying the measurement site with which the electrode 2 is to be brought into contact in accordance with the set lead type on the touch panel display 202 of the smartphone 200 allows the subject to bring the electrode 2 into contact with the correct position. Guiding the subject to the measurement site by such a guide screen 2024 allows the optimal lead to be more reliably set, and the electrocardiographic waveform to be correctly measured. Here, the buttons 2022 including the buttons 2022a to 2022i correspond to the setting unit of the present invention.

The lead type set in step S604 is transmitted from the smartphone 200 to the portable electrocardiographic device 100. The portable electrocardiographic device 100 receives the lead type (step S503), and stores the same in a predetermined region of the memory unit 106.

Next, in the portable electrocardiographic device 100, the control unit 104 detects the electrode contact state (step S504). Specifically, when measurement with lead I is performed with the portable electrocardiographic device 100, the tip of the right hand index finger is brought into contact with the second electrode 3, and the middle phalanx of the right hand index finger is brought into contact with the third electrode 4. Then, the left palm is brought into contact with the first electrode 2. As described above, the subject respectively brings the electrodes 2, 3, and 4 into contact with the measurement sites in accordance with the set lead type. Electrical signals respectively acquired via the electrodes 2, 3, and 4 are amplified in the amplifier unit 102 and digitally converted in the AD conversion unit 103 to generate a contact state detection signal. The contact state detection signals generated in this manner are transmitted to the control unit 104, and the contact states between the subject and each of the electrodes 2, 3, and 4 are detected.

In the portable electrocardiographic device 100, information indicating the electrode contact state is transmitted to the smartphone 200 (step S505). Upon receiving the information indicating the electrode contact state (step S605), the smartphone 200 displays the electrode contact state on the touch panel display 202 and the like (step S606) to notify the subject that normal contact is maintained with each of the electrodes 2, 3, and 4.

The control unit 104 determines whether a predetermined time has elapsed with the electrode contact state being maintained (step S506).

If a “NO” determination is made in step S506, the processing returns to step S504.
If a “YES” determination is made in step S506, the control unit 104 starts electrocardiographic measurement in the set lead I (step S507).

Once electrocardiographic measurement is started, the portable electrocardiographic device 100 performs streaming communication to and from the smartphone 200, and transmits lead type information indicating that the lead type is lead I, electrocardiographic waveform information, and measurement time information to the smartphone 200 (step S508). The measurement time information is information related to the time elapsed since the start of electrocardiographic measurement, which is counted in the timer unit 105. Here, the measurement time information is information indicating the remaining measurement time obtained by subtracting, from a predetermined time, the time elapsed since the start of electrocardiographic measurement. The information on the time elapsed since the start of electrocardiographic measurement may be transmitted from the portable electrocardiographic device 100 to the smartphone 200 to perform the processing of subtracting the same from the predetermined time on the smartphone 200 side. On the other hand, the smartphone 200 receives the lead type information, the electrocardiographic waveform information, and the measurement time information from the portable electrocardiographic device 100 (step S607).

The smartphone 200 displays the lead type, the electrocardiographic waveform, and the measurement time on the touch panel display 202 (step S608). In this way, the subject is notified that the lead type is lead I, that the electrocardiographic measurement is being normally performed, and of the remaining measurement time.

Whether a predetermined measurement time (e.g., 30 seconds) has elapsed since the start of electrocardiographic waveform measurement is determined (step S509).

If a “NO” determination is made in step S509, the processing returns to step S507 to continue electrocardiographic measurement.
If a “YES” determination is made in step S509, the control unit 104 analyzes the electrocardiographic waveform in accordance with the set predetermined lead system (step S510). Analyzing the electrocardiographic waveform in accordance with the set lead I allows accurate analysis.

During electrocardiographic waveform analysis, the control unit 104 transmits information indicating that electrocardiographic waveform analysis is in execution to the smartphone 200 (step S511). Upon receiving the information indicating that electrocardiographic waveform analysis is in execution from the portable electrocardiographic device 100 (step S609), the smartphone 200 displays information indicating that electrocardiographic waveform analysis is in execution on the touch panel display 202 (step S610).

Upon completion of electrocardiographic waveform analysis, the control unit 104 stores the lead type, which is lead I, the electrocardiographic waveform, and the analysis result in association with one another in a predetermined region of the memory unit 106 (step S512). Storing the lead type in association with the electrocardiographic waveform and the analysis result in the predetermined region of the memory unit 106 allows useful information to be provided when the physician reads out the electrocardiographic waveform and utilizes the same for diagnosis or the like. The lead type, the electrocardiographic waveform, and the analysis result associated with one another may be stored only on the smartphone 200 side without being stored in the memory unit 106 of the portable electrocardiographic device 100. Furthermore, only one of the lead type, the electrocardiographic waveform, and the analysis result may be stored in the memory unit 106 of the portable electrocardiographic device 100.

When an abnormal waveform is detected in electrocardiographic waveform analysis, the control unit 104 may cause an abnormal waveform detection LED 13 to blink to notify the subject of the abnormal waveform detection.

Furthermore, upon completion of electrocardiographic waveform analysis, the control unit 104 transmits the analysis result to the smartphone 200 by high-speed data communication (step S514). At this time, the smartphone 200 receives the analysis result transmitted from the portable electrocardiographic device 100 (step S611), and displays the analysis result, that is, whether the electrocardiographic measurement result is normal and without any problems or whether an abnormal waveform has been detected, on the touch panel display 202 (step S612).

The control unit 104 determines, as a result of electrocardiographic waveform analysis, whether there is an abnormality in the electrocardiographic waveform with lead I (step S515).

If a “NO” determination is made in step S515, the electrocardiographic measurement processing is terminated. Depressing the power switch 7 turns off the power (step S516).
If a “YES” determination is made in step S515, that is, if an abnormality is observed in the electrocardiographic waveform with lead I, the control unit 104 transmits the addition of remeasurement in another lead system to the smartphone 200 to perform electrocardiographic measurement in a more correct lead system (step S517).

An example of the analysis result displayed on the touch panel display 202 when it is determined that there is an abnormality in the electrocardiographic waveform with lead I in step S515 is illustrated in FIG. 13(B). Here, the analysis result of the electrocardiographic waveform with lead I is displayed on the touch panel display 202. An analysis result indication 2025 that states “Arrhythmia is observed,” and an indication 2026 prompting for electrocardiographic waveform remeasurement in a different lead system that states “Perform additional lead V4 measurement for correct diagnosis” are presented on the touch panel display 202. As described above, the control unit 104 corresponds to the remeasurement facilitating unit of the present invention, which determines whether there is an abnormality in the electrocardiographic waveform; that, when it is determined that there is an abnormality, transmits the addition of remeasurement in another lead system to the smartphone 200; and that causes the touch panel display 202 to present the indication 2026 prompting for electrocardiographic waveform remeasurement in a different lead system. Furthermore, here, the touch panel display 202, which presents the indication 2026 prompting for electrocardiographic waveform remeasurement in a different lead system, corresponds to the display unit of the present invention.

As illustrated in FIG. 6(A), in electrocardiographic waveforms with lead I, whether there is an irregular pulse wave can be approximately determined by the interval between R waves, which have high peak values. However, the sizes of PQRST waves, which are typical electrocardiographic waveform parameters illustrated in FIG. 5, are small, and thus the optimal analysis is difficult. Therefore, terminating electrocardiographic waveform measurement at this stage means ending with a simplified electrocardiographic waveform measurement with lead I without enabling more accurate electrocardiographic waveform measurement and analysis. Accordingly, in electrocardiographic waveform measurement with lead I, when an abnormal electrocardiographic waveform such as arrhythmia is detected or when the waveform quality is defective, the indication 2026 prompting for electrocardiographic waveform measurement in another lead system is presented on the touch panel display 202 of the smartphone 200 (step S613), as illustrated in FIG. 13(B). Here, to more precisely grasp and analyze the electrocardiographic waveform pattern, a prompt for electrocardiographic measurement with lead V4 is performed.

When the subject or the user touches the touch panel display 202 on which the indication 2026 prompting for electrocardiographic waveform measurement in another lead system illustrated in FIG. 13(B) is presented, the lead type setting screen 2021 illustrated in FIG. 10(A) is displayed. When the subject or the user touches the button 2022g for setting lead V4, the consent (OK) of the subject or the user to the addition of electrocardiographic waveform remeasurement with lead V4, which is another lead system, is acquired (step S614). In response to this, as information on another lead type, information indicating that another lead type is lead V4 is transmitted from the smartphone 200 to the portable electrocardiographic device 100. Furthermore, when the subject or the user touches the button 2022g for setting lead V4 on the lead type setting screen 2021, the touch panel display 202 displays the guide screen 2023 that describes, using a figure and characters, the position (measurement site) with which the subject is to bring the electrode 2 of the portable electrocardiographic device 100 into contact in accordance with the set lead system, as illustrated in FIG. 10(B). This allows the subject to bring the electrode 2 into contact with the correct position, and the electrocardiographic waveform to be correctly measured.

The portable electrocardiographic device 100 receives information on measurement with another lead (step S518). Thereafter, electrocardiographic measurement with lead V4, which has been set as another lead system, is performed. The processing procedures of step S519 and beyond are the same as the processing procedures of step S304 and step S405 and beyond illustrated in FIG. 8 and FIG. 9, and thus description thereof is omitted.

The lead system added to the electrocardiographic waveform measurement with lead I is not limited to lead V4 described above, and various lead systems can be set. For example, when the control unit 104 determines, as a result of electrocardiographic waveform analysis in step S518, that there is a possibility of atrial fibrillation (AF), it is difficult to make a more reliable determination on atrial fibrillation with lead I, and it is preferable to check the presence or absence of P waves or F waves (irregular baseline fluctuations). In this case, electrocardiographic waveform remeasurement with lead V1 is added in step S32. The electrocardiographic waveform with lead V1 is a waveform illustrated in FIG. 6(D). Thus, additionally performing remeasurement with lead V1, in which P waves and F waves are easily grasped, allows electrocardiographic waveform data to be collected that is more beneficial in determining the presence or absence of atrial fibrillation.

In this way, in addition to electrocardiographic measurement in one lead system, electrocardiographic waveform remeasurement in another lead system can be performed. This allows the electrocardiographic waveform pattern to be correctly measured, and information beneficial for correct diagnosis to be collected. An example in which electrocardiographic waveform remeasurement with lead V4 is added to electrocardiographic measurement with lead I, and an example in which electrocardiographic waveform remeasurement with lead V1 is added to the electrocardiographic measurement with lead I have been described. However, lead systems used when performing electrocardiographic waveform remeasurement in addition to the electrocardiographic measurement with lead I is not limited thereto. The combination of the lead system when performing the initial electrocardiographic measurement and the lead system when electrocardiographic waveform remeasurement is added is not limited thereto either. When accurate analysis cannot be expected in the electrocardiographic measurement with one lead system for reasons such as poor waveform quality, much noise, and unclear waveform pattern, electrocardiographic waveform remeasurement with a lead system, in which an electrocardiographic waveform having a property complementary to that of the electrocardiographic waveform with the one lead system can be measured, can be added to improve the accuracy of electrocardiographic measurement.

### REFERENCE NUMERALS LIST

1: Portable electrocardiographic device main body
2, 3, 4: Electrode
13: Lead type setting input unit
14: Lead type display LED
100: Portable electrocardiographic device
200: Smartphone
202: Touch panel display

What is claimed is:

1. A portable electrocardiographic device configured to measure an electrocardiographic waveform using a plurality of types of lead systems, the portable electrocardiographic device comprising:
an electrode unit configured to be brought into contact with a predetermined location of a subject's body and measure an electrocardiographic waveform;
an analysis unit configured to analyze the electrocardiographic waveform measured by the electrode unit in accordance with a lead system at a time of measurement of the electrocardiographic waveform;
a storage unit configured to store the electrocardiographic waveform measured at the electrode unit, the lead system, and an analysis result of the electrocardiographic waveform analyzed by the analysis unit in association with one another; and
a remeasurement facilitating unit configured to prompt a user, when the analysis result or a state of the measured electrocardiographic waveform satisfies a predetermined condition, for remeasurement in a predetermined lead system different from the lead system at the time of the measurement of the electrocardiographic waveform.

2. The portable electrocardiographic device according to claim 1, wherein the remeasurement facilitating unit includes a display unit configured to display the lead system to be set at a time of the remeasurement.

3. The portable electrocardiographic device according to claim 2, wherein the display unit further displays indicating that the predetermined condition is satisfied.

4. The portable electrocardiographic device according to claim 1, further comprising:
a setting unit configured to set lead system used in measurement of the electrocardiographic waveform, among the plurality of types of lead systems, wherein
at the time of the measurement and at a time of the remeasurement, the user sets the lead system using the setting unit.

5. The portable electrocardiographic device according to claim 1, wherein
the lead system at the time of the measurement is lead I in a 12-lead system,
the predetermined condition is that arrhythmia is observed in the analysis result, and
the predetermined lead system is lead V4 in the 12-lead system.

6. The portable electrocardiographic device according to claim 1, wherein
the lead system at the time of the measurement is lead I in a 12-lead system,
the predetermined condition is that atrial fibrillation is observed in the analysis result, and
the predetermined lead system is lead V1 in the 12-lead system.

7. The portable electrocardiographic device according to claim 1, wherein
the lead system at the time of the measurement is lead I in a 12-lead system,
the predetermined condition is that a defect in waveform quality is observed in the analysis result, and
the predetermined lead system is lead V1 or lead V4 in the 12-lead system.

8. An electrocardiographic measurement system configured to measure an electrocardiographic waveform using a plurality of types of lead systems, the electrocardiographic measurement system comprising:
a portable electrocardiographic device provided with an electrode unit configured to be brought into contact with a predetermined location of a subject's body and detect an electrocardiographic waveform; and
a portable communication terminal provided communicably with the portable electrocardiographic device, wherein
the electrocardiographic measurement system further includes
an analysis unit configured to analyze the electrocardiographic waveform measured by the electrode unit in accordance with a lead system at a time of measurement of the electrocardiographic waveform,
a storage unit configured to store the electrocardiographic waveform measured at the electrode unit, the lead system, and an analysis result of the electrocardiographic waveform analyzed by the analysis unit in association with one another, and
a remeasurement facilitating unit configured to prompt a user, when the analysis result or a state of the measured electrocardiographic waveform satisfies a predetermined condition, for remeasurement in a predetermined lead system different from the lead system at the time of the measurement of the electrocardiographic waveform.

9. The electrocardiographic measurement system according to claim 8, wherein
the remeasurement facilitating unit includes a display unit provided at either the portable electrocardiographic device or the portable communication terminal, the display unit is configured to display the lead system to be set at a time of the remeasurement.

10. The electrocardiographic measurement system according to claim 9, wherein the display unit further displays indicating that the predetermined condition is satisfied.

11. The electrocardiographic measurement system according to claim 8, further comprising:
a setting unit configured to set lead system used in measurement of the electrocardiographic waveform, among the plurality of types of lead systems, wherein
at the time of the measurement and at a time of the remeasurement, the user sets the lead system using the setting unit.

12. The electrocardiographic measurement system according to claim 8, wherein
the lead system at the time of the measurement is lead I in a 12-lead system,
the predetermined condition is that arrhythmia is observed in the analysis result, and
the predetermined lead system is lead V4 in the 12-lead system.

13. The electrocardiographic measurement system according to claim 8, wherein
the lead system at the time of the measurement is lead I in a 12-lead system,
the predetermined condition is that atrial fibrillation is observed in the analysis result, and
the predetermined lead system is lead V1 in the 12-lead system.

14. The electrocardiographic measurement system according to claim 8, wherein
the lead system at the time of the measurement is lead I in a 12-lead system,
the predetermined condition is that a defect in waveform quality is observed in the analysis result, and
the predetermined lead system is lead V1 or lead V4 in the 12-lead system.

15. A non-transitory recording medium having a program recorded therein wherein
the display unit in the electrocardiographic measurement system according to claim 9 is provided at the portable communication terminal, and
the program is for controlling the portable communication terminal to cause the display unit to display the lead system to be set at the time of the remeasurement.

16. A non-transitory recording medium having a program recorded therein wherein
the display unit in the electrocardiographic measurement system according to claim 10 is provided at the portable communication terminal, and
the program is for controlling the portable communication terminal to cause the display unit to display indicating that the predetermined condition is satisfied.

17. A non-transitory recording medium having a program recorded therein wherein
the setting unit in the electrocardiographic measurement system according to claim 11 is provided at the portable communication terminal, and
the program is for controlling the portable communication terminal to allow the user to set the lead system using the setting unit at the time of the measurement and at the time of the remeasurement.

18. The portable electrocardiographic device according to claim 2, further comprising:
a setting unit configured to set lead system used in measurement of the electrocardiographic waveform, among the plurality of types of lead systems, wherein
at the time of the measurement and at the time of the remeasurement, the user sets the lead system using the setting unit.

19. The portable electrocardiographic device according to claim 3, further comprising:
a setting unit configured to set lead system used in measurement of the electrocardiographic waveform, among the plurality of types of lead systems, wherein
at the time of the measurement and at the time of the remeasurement, the user sets the lead system using the setting unit.

20. The portable electrocardiographic device according to claim 2, wherein
the lead system at the time of the measurement is lead I in a 12-lead system,
the predetermined condition is that arrhythmia is observed in the analysis result, and
the predetermined lead system is lead V4 in the 12-lead system.
